(19) United States
(12) Patent Application Publication
(10) Pub. No.: US 2008/0255236 A1
(43) Pub. Date: Oct. 16, 2008

(54) Methods and Composition Comprising 2-[(3-Chlorophenyl)Amino] Phenylacetic Acid for Alzheimer's Disease

(75) Inventors: Margo P. Cohen, New York, NY (US); Clyde W. Shearman, Westchester, PA (US); Gregory T. Lautenslager, Oaklyn, NJ (US)

Correspondence Address: MORGAN, LEWIS & BOCKIUS LLP, 1701 MARKET STREET, PHILADELPHIA, PA, 19103-2921, US

(73) Assignee: Glycadia, Inc., Philadelphia, PA (US)

(21) Appl. No.: 12/100,119

(22) Filed: Apr. 9, 2008

Related U.S. Application Data
(60) Provisional application No. 60/911,399, filed on Apr. 12, 2007.

(51) Int. Cl. A61K 31/196; A61P 25/28

(52) U.S. Cl. 514/567

(57) ABSTRACT

The present invention is directed to a method of preventing or arresting the progression of Alzheimer's disease by administering to a patient having Alzheimer's disease a composition comprising an amount of 2-[(3-chlorophenyl)amino]phenylacetic acid, or a pharmaceutically acceptable salt thereof, sufficient to elicit a prophylactic or therapeutic effect. In some embodiments, the composition is administered orally, transdermally, intravenously, intrathecally, or by suppository. In some embodiments, the composition comprises a pharmaceutically acceptable carrier.

### CROSS-REFERENCE TO RELATED APPLICATIONS

This application claims the benefit of U.S. Provisional Application No. 60/911,399 filed Apr. 12, 2007, which is incorporated herein by reference, in its entirety.

### BACKGROUND OF THE INVENTION

The present invention is directed to compositions that inhibit the formation of aggregate-inducing amyloid β (Aβ) peptides generated from the amyloid β protein precursor (APP) that are causally implicated in the development of Alzheimer's disease, as well as methods of use of these compositions for the prevention or arrest of progression of Alzheimer's disease. More particularly, it has been discovered that the anti-glycation compound 2-[(3-chlorophenyl)amino]phenylacetic acid (23CPPA) reduces the production of aggregate-inducing Aβ peptides in mammalian cells and provides a method of preventing or arresting the development of Alzheimer's disease. The method includes the step of administering to a patient in need of such treatment a composition comprising the above compound or a pharmaceutically acceptable salt thereof in an amount sufficient to elicit a prophylactic or therapeutic effect.

This invention relates to the use of 2-[(3-chlorophenyl)amino]phenylacetic acid in the prevention and treatment of Alzheimer's disease.

23CPPA, 2-[(3-chlorophenyl)amino]phenylacetic acid, is an anti-glycation agent that interacts with the binding pocket domains IIA and IIIA in the albumin molecule, rendering susceptible lysine amino groups in or near the binding pockets inaccessible for condensation with glucose in a reaction known as nonenzymatic glycation (U.S. Pat. Nos. 6,355,680 and 6,552,077). Chronic administration of 23CPPA to diabetic rodents lowers the concentration of glycated albumin in the face of persistent hyperglycemia, and ameliorates the structural and functional abnormalities characteristic of diabetic kidney disease (Cohen et al: Kid Int 61:2025-2032, 2002; 68:1554-1561,2005; AJP Renal:292:789-795, 2007). 23CPPA does not have the molecular formula of a nonsteroidal anti-inflammatory (NSAID) drug, is not an isomer or enantiomer of a NSAID, and is not a pharmacologic inhibitor of the cyclooxygenase enzymes COX1 and COX2.

Alzheimer's disease, a neurodegenerative disease involving parts of the brain that control thought, memory and language, is the most common form of age-related dementia, affecting an estimated five million people in the United States. Patients with Alzheimer's disease have a progressive loss of mental and physical function, with clinical manifestations that include agitation, indifference, disorientation, memory loss, delusional thinking, inability to verbalize thoughts, language deterioration, and eventual aphasia, disablement, and immobility. Pathological manifestations include abnormal clumps of protein, known as amyloid plaques, and tangled bundles of nerve fibers, known as neurofibrillary tangles. Gene mutations have been identified in some of the less than 10% of all patients with Alzheimer's disease in whom there is a familial tendency and the onset of Alzheimer's disease is relatively early (age 30 to 60 years), but the role of genes is less clear in the more common, sporadic form of Alzheimer's disease that affects 90% of patients in whom onset of the disease is later (age 65 years and older).

Neurofibrillary tangles in the brains of people with Alzheimer's disease are marked by the presence of tau, a microtubule-associated protein that normally functions to promote tubulin assembly, microtubule stability and cytoskeletal assembly (Shahani: Cell Mol Life Sci 59:1668-1680, 2002). Paired helical filament tau is the principal neurofibrillary component. The tau that accumulates in Alzheimer's disease lesions differs from microtubule-associated-protein in its state of aggregation and post-translational modification, including levels of phosphorylation, glycation, proteolytic truncation and racemization (Goedert: Ann NY Acad Sci 777:121-131, 1996; Ledesma et al: J Neurochem 65:1658-1664, 1995; Mena et al: Acta Neuropathol 91:633-641, 1996; Watanabe et al: J Biol Chem 274:7368-7378, 1999). Phosphorylation and nonenzymatic glycation can decrease the affinity of tau for tubulin and its microtubule binding activity, and have been implicated in events leading to tau aggregation, fibrillization and neuronal death (Li et al: J Biol Chem 279:15938-15945, 2004; Yan et al: Proc Natl Acad Sci 91:7787-7791, 1994; Yan et al: Nat Med 1:693-699, 1995; Ledesma et al: J Biol Chem 269:21614-21619, 1994; Necula & Kuret: J Biol Chem 279:49694-49703, 2004).

By favoring the induction of aberrant cleavage of the amyloid β protein precursor (APP) that gives rise to toxic Aβ species, the nonenzymatic glycation of amyloid β protein precursor may contribute to the aggregation and deposition of Aβ peptides found in amyloid plaques in the brains of people with Alzheimer's disease (Schmitt: Med Hypotheses 66:898-906, 2006). Moreover, the nonenzymatic glycation of albumin induces the formation of dimers, multimers, amorphous aggregates and sheet-like fibers that exhibit the amyloid fiber-specific quarternary structure element known as amyloid fiber cross-β structure (Bouma et al: J Biol Chem 278:43:41810-41819, 2003). These observations suggest that glycated albumin may play a more direct role in the pathogenesis of Alzheimer's disease. Indeed, this possibility is supported by the finding that the cerebrospinal fluid level of nonenzymatically glycated albumin is increased about 1.7 fold in Alzheimer's disease patients compared to that in age-matched non-demented controls (Shuvaev et al: Neurobiol Aging 22:397-402, 2001). Inhibiting the nonenzymatic condensation of glucose with albumin with the anti-glycation compound 23CPPA may create a more favorable balance in Aβ peptides resulting from APP cleavage.

Amyloid plaques in the brains of people with Alzheimer's disease are marked by an accumulation of amyloid β (Aβ) protein, an approximately 4-kilodalton protein that is believed to play a causal role in the development of this disease. The accumulation of Aβ in the brain is thought to play a central role in the pathology of Alzheimer's disease, instigating a pathological cascade that ultimately results in neuronal dysfunction and death (Selkoe: Physiol Rev 81:741-766, 2001; Hardy & Higgins: Science 256:184-185, 1992). The amyloid cascade hypothesis encompasses experimental data indicating that multiple Amyloid p (Aβ) species with varying amino and carboxytermini are generated from the amyloid β protein precursor (APP) through sequential proteolytic cleavages by the β and γ secretases (Golde et al: Biochim Biophys Acta 1502:172-187, 2000). Of particular interest among these Aβ species are the 40-amino acid form, Aβ40, which is the most abundantly produced Aβ peptide, and a slightly longer and less abundant 42-amino acid form, Aβ42. It has been suggested that there is an imbalance in production of these Aβ species, with a relative increase in Aβ42, in the pathologic amyloid cascade (Younkin: J Physiol Paris, 92:289-292, 1998). Aβ42 forms aggregates that are toxic to various cells in culture much more readily than does Aβ40 and other shorter Aβ peptides, and Aβ42 is deposited earlier and more consistently than Aβ40 in the brain of patients with Alzheimer's disease. There is a preferential increase the amount of Aβ42 produced, increasing the ratio of Aβ42 to Aβ40, in mutations associated with familial Alzheimer's disease (Selkoe: Physiol Rev 81:741-766, 2001; Scheuner et al: Nat Med 2:864-870, 1996). These observations have suggested that reducing the production of Aβ42 and/or increasing the ratio of Ab42 to Aβ40 may help prevent the development or progression of Alzheimer's disease.

Some NSAIDs have been to found to lower levels of the amyloidogenic Aβ42 peptide in medium from cultured cells and in brains of APP transgenic mice (Weggen et al: Nature 414:212-216, 2001; Eriksen et al: J Clin Invest 112:440-449, 2003). This property was thought to result from anti-inflammatory and COX-inhibitory activities of the NSAIDs. However, some NSAIDs may decrease Aβ42 production by mechanisms that do not require inhibition of cyclooxygenase enzymes, suggesting that intrinsic features of particular NSAIDs may have other activities that influence production of this amyloidogenic peptide, perhaps by affecting the γ-secretase complex responsible for proteolytic cleavages in the APP (Weggen et al: J Biol Chem 278:31831-31837, 2003; Sagi et al: J Biol Chem 278:31825-31830, 2003; Takahishi et al: J Biol Chem 278:18664-18760, 2003). γ-secretase is a multiprotein complex consisting of at least four membrane-bound proteins: presenilin, nicastrin, APH-1 and PEN-2, all of which are required for proper maturation and activity of the complex (McClendon et al: FASEB J 14:2383-2386, 2000; Murphy: J Biol Chem 275:26277-26284, 2000).

Regardless of the mechanism responsible for the Aβ42-lowering properties of NSAIDs, their clinical use in Alzheimer's disease is limited given the gastrointestinal side effects resulting from the inhibition of COX enzymes and the cardiotoxicity associated with selective COX-2 inhibition as well as with some older nonspecific COX-inhibitors. A compound possessing comparable Aβ42-lowering properties that does not exhibit pharmacological COX-inhibitory activity and therefore would not be associated with the gastrointestinal, cardiac and renal toxicities associated with chronic use of NSAIDs, particularly in an older population, would be an important addition to the clinical armamentarium for prevention and treatment of Alzheimer's disease. The R-enantiomer of flurbiprofen, which is said to lack COX-inhibitory activity, is purported to fulfill these criteria (Erikson et al: J Clin Invest 112:440-449, 2003). However, 22-30% of R-flurbiprofen is converted in vivo to the potent COX-inhibitory enantiomer S-flurbiprofen (Erikson et al: J Clin Invest 112:440-449, 2003; Wechter et al: Chirality 6:457-459, 1994). 23CPPA, in contrast, which lowers Aβ42 production and lacks intrinsic COX-inhibitory activity, is not converted to a compound with COX-inhibitory activity and better fulfills these criteria, affording safer protection against the development of Alzheimer's disease.

The ability of some but not all NSAIDs to reduce Aβ42 suggests that compound-specific non-cyclooxygenase activity is responsible for this property of lowering Aβ42 (Sagi et al: J Biol Chem 278:31825-31830, 2003). Since 2-[(3-chlorophenyl)amino]phenylacetic acid does not have the molecular formula of a nonsteroidal anti-inflammatory drug, is not an isomer or enantiomer of a NSAID, and is not a pharmacologic inhibitor of the cyclooxygenase enzymes COX1 and COX2, the ability of this compound to inhibit the formation of amyloidogenic peptides in mammalian cells is unexpected and not anticipated by one skilled in the art. The present invention demonstrates that 2-[(3-chlorophenyl)amino]phenylacetic acid possesses an intrinsic and compound-specific ability to influence γ-secretase function, by either affecting the enzymatic machinery or its substrate, and the production of Aβ42 in mammalian cells.

### SUMMARY OF THE INVENTION

The present invention provides novel methods and compositions that inhibit the formation of aggregate-inducing amyloid β (Aβ) peptides generated from the amyloid β protein precursor (APP) that are causally implicated in the development of Alzheimer's disease.

The present invention also provides novel methods and compositions for the prevention and arrest of progression of Alzheimer's disease. The method includes the step of administering to a patient in need of such treatment a composition comprising the above compound or a pharmaceutically acceptable salt thereof in an amount sufficient to elicit a prophylactic or therapeutic effect.

In some embodiments, these and other aspects of the invention are achieved with the discovery that the anti-glycation compound 2-[(3-chlorophenyl)amino]phenylacetic acid (23CPPA) reduces the production of aggregate-inducing Aβ peptides in mammalian cells and thereby may prevent or arrest the development of Alzheimer's disease.

### BRIEF DESCRIPTION OF THE DRAWINGS

Further aspects of the instant invention will be more readily appreciated upon review of the detailed description of the preferred embodiments included below when taken in conjunction with the accompanying drawings, of which:

FIG. 1 is a chart diagram representing an exemplary embodiment of 23CPPA lowering production of amyloidogenic Aβ42 peptide. Mammalian cell cultures expressing human Amyloid Protein Precursor (APP) were treated with the sodium salt of 23CPPA at concentrations of 10, 50 and 100 micromolar. The culture supernatants were analyzed by enzyme-linked immunoassay for the Aβ40 and Aβ42 peptides. Results were compared to control values obtained from cells treated with DMSO. Treatment with 23CPPA produced a dose-dependent reduction in the amount of Aβ42 without proportionate change in the amount of Aβ40 or of total Aβ values.

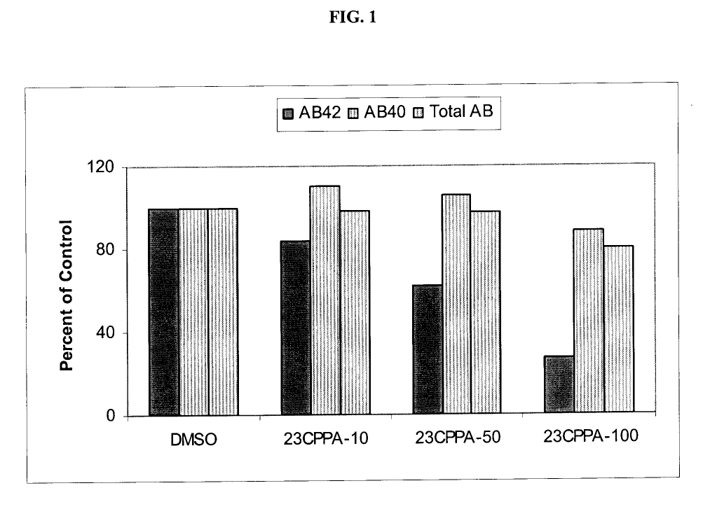

### DETAILED DESCRIPTION OF THE INVENTION

It has been surprisingly discovered as described in the present invention that 23CPPA reduces the production of aggregate-inducing amyloid β (Aβ) peptides in mammalian cells and therefore provides a method of preventing or arresting the development of Alzheimer's disease.

It is a novel and unanticipated finding of the present invention that the compound 23CPPA and its pharmaceutically acceptable salts, which inhibit the nonenzymatic glycation of albumin and are useful in the treatment of glycation-related pathologies in diabetes, possess the intrinsic ability to lower the production in mammalian cells of aggregate-inducing amyloid β (Aβ peptides generated from the amyloid β protein precursor (APP) that are causally implicated in the development of Alzheimer's disease.

The compound(s) of the present invention inhibit the generation of toxic peptides that are increased in the brain in Alzheimer's disease. Since therapeutic concentrations of the compound(s) of the present invention are capable of reducing the formation of the amyloidogenic Aβ42 peptide, the present invention provides a novel and improved method for the treatment of Alzheimer's disease.

This invention also provides therapeutic compositions comprising the above-described compound(s).

This invention further provides a method for treating Alzheimer's disease comprising administering to the patient an effective amount of a therapeutic composition comprised of the above-described compound(s) capable of reducing the formation of aggregate-inducing amyloid β (Aβ) peptides and a pharmaceutically acceptable carrier.

The present invention also comprises compounds as described above formulated into compositions together with one or more non-toxic physiologically acceptable carriers, adjuvants or vehicles which are collectively referred to herein as carriers, for parenteral injection, for oral administration in solid or liquid form, for rectal or topical administration, or the like. The compositions can be administered to humans either orally, rectally, parenterally (intravenously, intramuscularly, subcutaneously), intracisternally, intravaginally, intraperitoneally, intravesically, locally (powders, ointments or drops), or as a buccal or nasal spray.

Compositions suitable for parenteral injection may comprise physiologically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propyleneglycol polyethyleneglycol, glycerol and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride and the like. Prolonged absorption of the injectable pharmaceutical form can be brought about by the use of agents delaying absorption, for example, aluminum monostearate and gelatin.

Solid dosage forms for oral administration include capsules, tablets, pills, powders and granules. In such solid dosage forms, the active compound may be admixed with at least one inert customary, pharmaceutically acceptable carrier, excipients (or carrier) such as sodium citrate or dicalcium phosphate or (a) fillers or extenders, as for example, starches, lactose, sucrose, glucose, mannitol and silicic acid, (b) binders, as for example, carboxymethylcellulose, alginates, gelatin, polyvinylpyrrolidone, sucrose and acacia, (c) humectants, as for example, glycerol, (d) disintegrating agents, as for example, agar-agar, calcium carbonate, potato or tapioca starch, alginic acid, certain complex silicates and sodium carbonate, (e) solution retarders, as for example, paraffin, (f) absorption accelerators, as for example, quarternary ammonium compounds, (g) wetting agents, as for example, cetyl alcohol and glycerol monostearate, (h) adsorbents, as for example, kaolin and bentonite, and (i) lubricants, as for example, talc, calciuk stearate, magnesium stearate, solid polyethylene glycols, sodium lauryl sulfate or mixtures thereof. In the case of capsules, tablets and pills, the dosage forms may also comprise buffering agents.

Solid compositions of a similar type may also be employed as fillers in soft and hard-filled gelatin capsules using such excipients as lactose or milk sugar as well as high molecular weight polyethylene glycols, and the like. Solid dosage forms such as tablets, capsules, pills and granules can be prepared with coatings and shells, such as enteric coatings and others well known in the art. They may contain opacifying agents, and can also be of such composition that they release the active compound or compounds in a certain part of the intestinal tract in a delayed manner. Examples of embedding compositions which can be used are polymeric substances and waxes.

The active compound(s) can also be in microencapsulated form, if appropriate, with one or more of the above-mentioned excipients.

Liquid dosage forms for oral administration include pharmaceutically acceptable emulsions, solutions suspensions, syrups and elixirs. In addition to the active compounds, the liquid dosage forms may contain inert diluents, commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan or mixtures of these substances, and the like.

Besides such inert diluents, the composition can also include adjuvants, such as wetting agents, emulsifying and suspending agents, sweetening, flavoring and perfuming agents.

Suspensions, in addition to the active compounds, may contain suspending agents, as for example, ethoxylated isostearyl alcohols, polyethylene sorbitol and sorbitan esters, microcrystalline cellulose, aluminum metahydrozide, bentonite, agar-agar and tragacanth, or mixtures of the substances, and the like.

Compositions for rectal or vaginal administration are preferably suppositories which can be prepared by mixing the compound(s) of the present invention with suitable non-irritating excipients or carriers such as cocoa butter, polyethyleneglycol or a suppository wax, which are solid at ordinary temperatures but liquid at body temperature and therefore, melt in the rectum or vagina cavity and release the active component.

Dosage forms for topical administration of a compound of this invention include ointments, powders, sprays and inhalants. The active component is admixed under sterile conditions with a physiologically acceptable carrier and any preservative, buffers or propellants as may be required. Ophthalmic formulations, eye ointments, powders and solutions are also contemplated as being within the scope of this invention.

Actual dosage levels of active ingredients in the compositions of the present invention may be varied so as to obtain an amount of active ingredient that is effective to obtain a desired therapeutic response for a particular composition and method of administration. The selected dosage level therefore depends upon the desired therapeutic effect, on the route of administration, on the desired duration of treatment and other factors.

The total daily dose of the compound(s) of this invention administered to a host in single or divided dose may be in amounts, for example, of 50 to about 1500 mg. Dosage unit compositions may contain such amounts or such submultiples therefore as may be used to make up the daily dose. It will be understood, however, that the specific dose level for any particular patient will depend on a variety of factors including body weight, general health, gender, diet, time and route of administration, rats of absorption and excretion, combination with other drugs, and the severity of the disease being treated. The dosage level may also depend on patient response as determined by measurement of one or more appropriate markers in suitable biological fluid or tissue at suitable intervals after administration.

The above disclosure generally describes the present invention. A more complete understanding can be obtained by reference to the following specific examples which are provided herein for purposes of illustration only, and are not intended to limit the scope of the invention. It should be appreciated by those of skill in the art, in light of the present disclosure, that many changes can be made in the specific embodiments which are disclosed and still obtain a like or similar results without departing from the spirit and scope of the invention.

The following examples are included to demonstrate embodiments of the invention.

### EXAMPLE 1

### Bioavailability of 23CPPA

Male rats were given a single dose of 23CPPA by the oral route (30 mg/kg) or by the intravenous route (3.0 mg/kg). Timed samples of blood were collected before and after dosing, and plasma concentrations of the compound were determined with LC-MS-MS analysis. Oral bioavailability, calculated from the plasma concentrations after oral versus intravenous administration, was 85%, indicating that the drug is absorbed from the gastrointestinal and enters the circulation for systemic delivery.

### EXAMPLE 2

### Plasma Levels of 23CPPA Following Oral Administration in Rats

Concentrations of compound in rat plasma were measured at timed intervals after administration by gavage of 25 and 75 mg per kilogram of body weight of the sodium salt of 23CPPA. Plasma levels of the compound increased after oral administration, reaching dose dependent maximal concentrations (Cmax) of 9500 ng per ml at 25 mg/kg and 25000 ng per ml at 75 mg/kg. Time of maximal concentration (Tₘₐₓ) was observed within one hour after administration. Plasma concentrations of the compound readily declined after reaching maximal concentration and the elimination half-life (T^{1/2}) was about 2 hours. The extent of systemic exposure of animals to 23CPPA, characterized by the area under the curve from 0 to 24 hours after administration (AUC₀₋₂₄), increased with increasing dosage, was approximately dose proportional, and encompassed levels representing therapeutic ranges.

| Dose (mg/kg body wt.) | Human Equivalent Dose | T^{1/2} | AUC₀₋₂₄ |
|---|---|---|---|
| 25 | 4.0 | 2.45 | 26600 |
| 75 | 12.5 | 1.95 | 94000 |

### EXAMPLE 3

### Brain levels of 23CPPA Following Oral Administration in Rats

Rat hemi brains were obtained 1, 4 and 6 hours after administration by gavage of the sodium salt of 23CPPA at doses of 15 and 60 mg/kg and were mixed with two equivalents of water and homogenized. The resulting homogenate was extracted into acetonitrile containing internal standard and concentrations of the compound were analyzed by high pressure liquid chromatography/two stage mass spectrometry (HPLC/MS/MS). Plasma samples also were collected from the same rats at these time points for measurement of plasma concentrations of the compound. Brain concentrations were dose proportional and showed an approximately 2:1 molar ratio with plasma concentrations, indicating excellent penetration of the compound into the brain.

| | Time | | |
|---|---|---|---|
| Dose (mg/kg) | after Dose | Brain (micromolar) | Blood (micromolar) |
| 15 | 1 hour | 76 | 27 |
| 15 | 4 hours | 12.4 | 4 |
| 15 | 6 hours | 11.9 | 3 |
| 60 | 1 hour | 148 | 57 |
| 60 | 4 hours | 61 | 28 |
| 60 | 6 hours | 45 | 22 |

### EXAMPLE 4

### Plasma Levels of 23CPPA After Oral Administration to Humans

Plasma concentrations were measured at timed intervals after oral administration of a single dose of 50 to 1000 mg of the sodium salt of 23CPPA. Plasma levels of the compound increased after oral administration, reaching dose dependent maximal concentrations of 200 to 15600 nanograms per ml. The mean time of maximal concentration (Tₘₐₓ) in all dosage groups was 1.74 hours. Plasma concentrations of the compound readily declined after reaching maximal concentration (Cmax) and the mean value for elimination half-life (T^{1/2}) was 1.77 hours. The extent of systemic exposure of subjects to 23CPPA, characterized by the area under the curve from 0 to 24 hours after administration (AUC₀₋₂₄), increased with increasing dosage from 50 to 1000 mg and was approximately dose proportional. The human pharmacokinetic findings closely resemble those in rats according to the human equivalent dose as shown in Example 2.

| Dose (mg) | mg/kg body wt | T^{1/2} | AUC₀₋₂₄ |
|---|---|---|---|
| 50 | 0.7 | 1.55 | 4480 |
| 100 | 1.3 | 1.50 | 11000 |
| 250 | 3.0 | 1.35 | 27260 |
| 500 | 6.0 | 1.50 | 54050 |
| 750 | 9.0 | 2.33 | 66650 |
| 1000 | 12.3 | 2.38 | 100430 |

### EXAMPLE 5

### 23CPPA Lowers Production of Amyloidogenic Aβ42 Peptide

Mammalian cell cultures expressing human Amyloid Protein Precursor (APP) were treated with the sodium salt of 23CPPA at concentrations of 10, 50 and 100 micromolar. The culture supernatants were analyzed by enzyme-linked immunoassay for the Aβ40 and Aβ42 peptides. Results were compared to control values obtained from cells treated with DMSO. Treatment with 23CPPA produced a dose-dependent reduction in the amount of Aβ42 without proportionate change in the amount of Aβ40 or of total Aβ values.

### EXAMPLE 6

### 23CPPA is Not Converted to COX-Inhibitory Compound

After incubation of 23CPPA with human hepatocytes, samples were analyzed using mass spectrometry to establish metabolic conversion of the compound. The conversion products identified consisted of hydroxylation and loss of water products and glucuronide conjugates, none of which had structures compatible with COX-inhibitory activity.

What is claimed is:

1. A method of preventing or arresting the progression of Alzheimer's Disease comprising the step of administering to a patient having Alzheimer's disease a composition comprising an amount of 2-[(3-chlorophenyl)amino]phenylacetic acid or a pharmaceutically acceptable salt thereof sufficient to elicit a prophylactic or therapeutic effect.

2. A method according to claim 1 wherein said composition is administered orally, transdermally, intravenously, intrathecally, or by suppository.

3. A method according to claim 1 wherein said composition comprises a pharmaceutically acceptable carrier.

4. A method according to claim 2 wherein said composition comprises a pharmaceutically acceptable carrier.
